(19) United States
(12) Patent Application Publication
(10) Pub. No.: US 2010/0160548 A1
(43) Pub. Date: Jun. 24, 2010

(54) PROCESS FOR THE PRODUCTION OF A DIANHYDROHEXITOL BASED POLYESTER

(76) Inventors: Bart Adrianus Johannes Noordover, Eindhover (NL); Rafael Jean Sablong, Eindhoven (NL); Robert Duchateau, Eindhoven (NL); Rudolfus Antonius Theodorus Maria van Benthem, Eindhoven (NL); Weihua Ming, Durham, NH (US); Cornelis Koning, Eindhoven (NL); Jacco van Haveren, Eindhoven (NL)

Correspondence Address: NIXON & VANDERHYE, PC, 901 NORTH GLEBE ROAD, 11TH FLOOR, ARLINGTON, VA, 22203, US

(21) Appl. No.: 12/441,474

(22) PCT Filed: Sep. 13, 2007
(86) PCT No.: PCT/EP07/07979

§ 371 Date: Feb. 1, 2010

(30) Foreign Application Priority Data
Sep. 15, 2006 (EP) PCT/EP2006/009011

(51) Int. Cl. C08G 63/12; C08G 63/91; C07D 493/04; C08L 67/00

(52) U.S. Cl. 524/601; 528/365; 525/444; 549/464; 525/440.15

(57) ABSTRACT

Process for the production of a polyester by the polycondensation of a mixture comprising isoidide, and a dicarboxylic acid or dicarboxylic acid anhydride, wherein the reaction is performed in the melt of the monomers and wherein these monomers are not activated. The polyesters based on one or more of the three isomers of dianhydrohexitol, being isosorbide, isomannide and isoidide, have properties which makes them suitable to be used in powder coatings, toner compositions as well as engineering plastics. The polyesters include a polyester according to the following formula, wherein n ranges from 3 to 300.

The invention relates to a process for the production of a polyester by the polycondensation of dianhydrohexitol and a dicarboxylic acid. The invention further relates to a polyester obtainable by the process according to the present invention. It also relates to a binder composition, a coating composition and a toner composition, all comprising the polyester obtainable by the process according to the present invention. Further the invention is related to an engineering plastic based on a polyester obtainable by the process according to the present invention.

Polyesters on the basis of dianhydrohexitol are disclosed in U.S. Pat. No. 6,291,629 B1. These polyesters are prepared by condensations between activated monomers, i.e. activated dicarboxylic acids and/or activated diols. The use of such activated monomers requires an activation step prior to the condensation step. An example of such a polycondensation is a condensation between a free diol and the dichloride of a dicarboxylic acid, the so-called HCL process. Another example is the so-called silyl process, a condensation between a bissilylated diol and the dichloride of a dicarboxylic acid. A third example is a transesterification process, a condensation between acetylated dianhydrohexitols and a free dicarboxylic acid. Such conventional types of polycondensations are further described inter alia by H. R. Kricheldorf and N. Probst in Macromol. Rapid. Commun. 16, 1995, 231, by N. Probst and H. R. Kricheldorf in High Perform. Polym. 7, 1995, 461 and by H. R. Kricheldorf, in J.M.S.—Rev. Macromol. Chem. Phys., 1997, C37, 599. The disadvantage of all these conventional types of polycondensations is that activated monomers are required.

U.S. Pat. No. 1,012,563 discloses the preparation of polyesters comprising isosorbide, a dicarboxylic acid and a diol, wherein the monomers are not activated. The use of isoidide is not described.

The purpose of the present invention is to provide a process for the production of a polyester based on isoidide and a dicarboxylic acid.

It is another object of the present invention to provide polyesters containing isoidide having improved properties.

It is a further object of the present invention to provide polyesters based on dianhydrohexitols that can be used in coating applications.

The invention relates to a process for the production of a polyester by the polycondensation of a mixture comprising isoidide, and a dicarboxylic acid or dicarboxylic acid anhydride, wherein the reaction is performed in the melt of the monomers and wherein these monomers are not activated.

Activated monomers are understood to be monomers that have been chemically modified, such as for example by reactions adding silyl groups or Cl-atoms to the monomers. A condensation reaction of a dicarboxylic acid to obtain an dicarboxylic acid anhydride as monomer is not considered to be an activation for the purpose of the present invention.

An advantage of the process according to the present invention is that no additional activation step is required for the condensation between dianhydrohexitol and a dicarboxylic acid or dicarboxylic acid anhydride. Very advantageous is that the process according to the present invention yields polyesters suitable to be used in powder coatings, toner compositions as well as engineering plastics. Further the process according to the present invention yields polyesters substantially colorless to colorless, an advantage which is very important for coating applications as well as for engineering plastics applications.

The process according to the present invention is performed in the melt of the monomers. Preferably, this condensation in the melt is performed at a temperature between 150 and 250° C. More preferable, the melt condensation is performed at a temperature of 180° C. or higher. A preferred temperature is one which is high enough to force the formation of ester bonds from carboxylic acids and diols, but not as high that thermal degradation and discoloration occurs.

The pressure applied in the process according to the present invention is not critical. In general the process is performed at atmospheric pressure, but optionally the pressure can be reduced. The use of a reduced pressure is advantageous in order to remove condensation products such as water and to obtain high molar weight polyesters. Typical high molar weight polyesters have number average molecular weights exceeding 10,000 g/mol. Preferably, the reduced pressure is a pressure below 50,000 Pa. More preferably the reduced pressure has a value between 10 and 5000 Pa. Most preferably between 100 and 500 Pa.

In order to remove condensation products such as water the reaction vessel may be flushed with an inert gas. In that case the setup is preferably continuously flushed with an inert gas. In general any inert gas can be used, but preferably, nitrogen is used.

Optionally a stabilizer may be added to the melt of the non-activated monomers. Examples of suitable stabilizers are phenolic stabilizers such as Irganox 259, Irganox 1010, Irganox 1330, Irganox B900, Irganox and Irganox HP2921 FF. It is also possible to add a mixture of two or more different stabilizers.

The process according to the present invention may be performed in the presence of an esterification catalyst. Suitable esterification catalysts include e.g. tetrabutyltitanate, tin(II) octoate, butyltinchloridedihydroxyide, manganese acetate, zinc acetate, para-toluene sulphonic acid. Titanium(IV) n-butoxide and tin(II)octoate are preferred esterification catalysts.

In the process according to the present invention in general a diol to diacid ratio of 1:1 is applied. If relatively low molecular weight polyesters are desired this ratio preferably deviates from such a 1:1 ratio by 0.1 to 0.2 units. Either an excess of diol or an excess of dicarboxylic acid may be used, respectively resulting in hydroxyl or carboxylic acid functional polyesters. Examples of such relatively low molecular weight polyesters are optionally curable polyesters for coating and toner applications.

In general any one of the three isomers of dianhydrohexitol may be used as non-activated dianhydrohexitol. The three isomers of dianhydrohexitol are isosorbide, isomannide and isoidide, respectively, having formula I, II and III, as presented below.

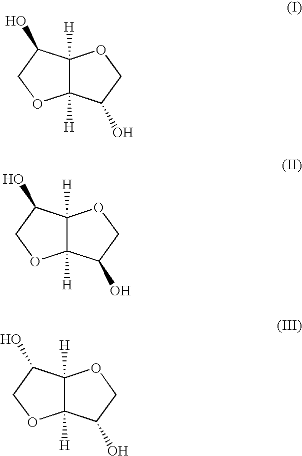

The isomers may be used alone or as a mixture of two or three of the isomers. However, surprisingly we have found that the use of isoidide is very advantageous. Polycondensations in the melt of non-activated isoidide and a non-activated dicarboxylic acid appeared to proceed faster than polycondensations with any one of the other two isomers. Thus, by using isoidide a lower condensation time and/or a lower temperature can be applied to obtain polyesters having required properties, such as a high molecular weight. This is very advantageous as it reduces the chance of thermal degradation and discoloration. Furthermore this is an industrial advantage from an economic point of view.

Preferably, the isomer used has a purity between 98% and 100%. More preferable the isomer has a purity above 99%. In particular the isomer has a purity above 99.5% and more in particular it has a purity above 99.8%. The higher the purity, the lower the discoloration will be. An added advantage of a higher purity is that polyesters with a higher molecular weight (i.e. M_{w}>25,000 g/mol) can be prepared.

The non-activated dicarboxylic acid may be any di- or polyvalent carboxylic acid. Examples of suitable di- or polyvalent carboxylic acids include maleic acid, fumaric acid, itaconic acid, citric acid, tartaric acid, citraconic acid, malonic acid, succinic acid, glutaric acid, adipic acid, sebacic acid, 1,4-cyclohexane dicarboxylic acid, hexahydrophthalic acid, hexachloroendomethylene tetrahydrophthalic acid, dichlorophthalic acid, isophthalic acid, terephthalic acid and/or trimellitic acid. Preferably, aliphatic di- or polyvalent carboxylic acids are being used. More preferable an aliphatic di- or polyvalent carboxylic acid having 4 to 20 carbon atoms is used as non-activated dicarboxylic acid. Most preferred is the use of linear aliphatic diacids such as succinic acid, glutaric acid, adipic acid and sebacic acid, having 4, 5, 6 and 10 carbon atoms, respectively.

Anhydrides of the non-activated dicarboxylic acids can also be used in the process of the present invention. Use of anhydrides gives the advantage of less formation of water in the polycondensation of the monomers to polymer.

For the process according to the present invention renewable as well as non-renewable monomers may be used. With renewable monomers are meant those starting materials that can be derived from natural products, growing in nature, contrary to rapidly reducing fossil resources. Examples of renewable monomers include succinic acid and citric acid. The use of renewable monomers provides non-fossil resource-derived polyesters.

The process according to the present invention makes it possible to prepare a polyester from isoidide and succinic acid, resulting in a polyester based on isoidide and succinic acid. A product which could not be prepared by a polycondensation of dianhydrohexitol and a dicarboxylic acid wherein the dicarboxylic acid is activated as dichloride or the diol is activated as acetylated diol, a condensation in solution as disclosed in patent U.S. Pat. No. 6,291,629 B1 as well as by Okada et al. in J. Appl. Pol. Sci., 1996, vol. 62, pages 2257-2265. Therefore the present invention also relates to the polyester based on isoidide and succinic acid units, having the following structure:

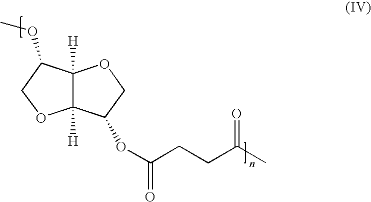

wherein n is an integer ranging from 3 to 300.

Of the dianhydrohexitol 1-99% may be replaced by another alcohol comprising two or more hydroxyl groups. Preferably, not more than 80%, more preferable not more than 60%, most preferable not more than 50% of the dianhydrohexitol may be replaced. In general any alcohol having two or more hydroxyl groups may be used to replace part of the dianhydrohexitol. Examples of suitable alcohols include glycerol, glycols, trimethylolpropane, pentaerythritol and aliphatic diols. Examples of suitable aliphatic diols include 1,3-propanediol, 1,4-butanediol, 2,3-butanediol, neopentyl glycol and 1,6-hexanediol. Preferably, an aliphatic diol having 2 to 10 carbon atoms is used. More preferable, an aliphatic alcohol is used having 2 to 6 carbon atoms such as 1,4-butanediol, 1,3-propanediol and 2,3-butanediol. 1,3-Propanediol is an example of a renewable alcohol.

The process according to the present invention is especially advantageous for the production of linear polyesters. The process according to the present invention may yield semi-crystalline polyesters. This is especially the case for polyesters with a regular chain structure, based on dianhydrohexitol and a linear dicarboxylic acid, such as e.g. isoidide and succinic acid (anhydride). Replacement of the dianhydrohexitol by another alcohol comprising two or more hydroxyl groups, as mentioned above, can be used to control the crystallinity of the polyesters obtainable by the process according to the present invention. In this way the semi-crystallinity can be used when required or suppressed where it is not required.

To the melt used in the process according to the present invention any suitable solvent may be added. Examples of suitable solvents are N-methyl pyrrolidone, dimethylformamide, dimethylacetamide, dimethylsulfoxide, and other relatively polar high boiling solvents. Preferably N-methylpyrrolidone, toluene or xylene is used as additional solvent.

Due to the rigidity of the bicyclic structure of dianhydrohexitol, the polyester is suitable to be used in a powder coating, e.g. a powder paint composition, in a toner composition as well to be used as an engineering plastic resin. Therefore the polyesters obtainable by the process according to the present invention can be used for all these applications.

For coating resins, functionalized polyesters having a relatively low molecular weight ranging from 1,500-6,000 g/mol (number averaged) are preferred.

Polyesters obtainable by the polycondensation of a mixture comprising a dianhydrohexitol, and a dicarboxylic acid or dicarboxylic acid anhydride, wherein the reaction is performed in the melt of the monomers and wherein these monomers are not activated, which can be applied in powder paint coating compositions yield in their non-cross linked form transparent, brittle coatings with a T_{g}: (glass transition temperature) above 40° C. Such powder paint coating compositions can be extruded with a crosslinker, yielding a binder composition. Suitable crosslinkers for coating applications are for example triglycidyl isocyanurate (TGIC) and N,N,N′,N′-tetrakis(2-hydroxyethyl)adipamide (Primid XL 552) for carboxylic acid functionalized polyesters, and the trimer of isophorone diisocyanate (Vestagon B 1530) and the trimer of hexamethylene diisocyanate (Desmodur N 3600) for hydroxy functionalized polyesters.

Optionally a catalyst and other additives such as a filler or pigment can be added. Coating compositions comprising one or more of the above-mentioned components can be extruded and subsequently ground to a fine powder. A typical particle size for such powder coating is <100 μm. Coating compositions comprising one or more of the above-mentioned components can also be applied to a substrate and subsequently cured. After crosslinking, transparent, tough and solvent resistant coatings are obtained.

Paper, wood, metal and plastic are just a few examples of many suitable substrates which can be partially or fully coated.

For engineering plastics high molecular weight polyesters are preferred. With high molecular weight is meant a number average molecular weight ranging from 5,000-100,000 g/mol. Isoidide is a preferred monomer for the preparation of an engineering plastic, as isoidide appeared to react faster with a non-activated dicarboxylic acid (anhydride).

Engineering plastics prepared by the process according to the present invention appeared to have high T_{g} values. Higher T_{g} values are advantageous for applications at elevated temperatures.

The invention will be elucidated with reference to the following examples, without being restricted by these.

### EXAMPLES

### Dianhydrohexitol

The isosorbide as well as the isoidide isomers having a purity of at least 98% were obtained from Roquette Frères (62080 Lestrem CEDEX France) and Agrotechnology and Food Innovations (P.O. Box 17, 6700 AA Wageningen, The Netherlands). Isoidide having a purity of at least 99.5% was obtained from Roquette Frères (62080 Lestrem CEDEX France).

### Experiment 1

### Synthesis of Polyester Based on Isosorbide and Succinic Acid

Succinic acid (44.9 g, 0.38 mol) and isosorbide (63.4 g, 0.43 mol) were weighed into a 250 mL round bottom glass flange reactor. The reactor was fitted with a vigreux column and a Dean-Stark type condenser to collect the condensation product. During the first part of the synthesis, the setup was continuously flushed with inert gas to limit oxidation and facilitate transport of water vapor. While stirring, the mixture was heated to 180° C. using a heating mantle. Titanium(IV) n-butoxide (0.02 mol % relative to succinic acid), dissolved in toluene, was added to the melt. Subsequently, the reaction temperature was increased stepwise to maintain distillation of the formed water. The maximum reaction temperature was 250° C. After 4 hours, vacuum processing was started at 230-250° C., with typical pressures ranging from 100-500 Pa. Vacuum was applied for 4 hours, after which the polymer was discharged from the reactor and left to cool and solidify. The resulting polyester 1 had a T_{g} value of 56.5° C., an Mₙ of 2400 g/mol, an acid value of 1.5 mg KOH/g and a hydroxyl value of 65.0 mgKOH/g.

### Example 2

### Synthesis of Polyester Based on Isoidide and Succinic Acid

Experiment 1 was repeated whereby succinic acid (3.37 g, 0.029 mol), isoidide (4.67 g, 0.032 mol) and Irganox HP2921 FF anti-oxidant (0.033 g) were weighed into a 50 mL three-necked round bottom glass reactor. The maximum reaction temperature was kept below 230° C. After 4 hours, vacuum processing was started at 230° C., with typical pressures ranging from 100-500 Pa. Vacuum was applied for 4 hours, after which the polymer was discharged from the reactor and left to cool and solidify. It was observed that this polyester, polyester 2, partly crystallizes from solution (solvent: CHCl₃) or during slow cooling from the melt. Yield: 92%. ¹H-NMR (ppm): 2.65 (m, 4H, succinic acid), 3.82-3.98 (m, 4H, H1, H6 isoidide), 4.62 (s, 2H, H3, H4 isoidide), 5.21 (d, 2H, H2, H5 isoidide). Mₙ=4200 g/mol (relative to PMMA standards), PDI=1.9. T_{g}=73.4° C., Tₘ=171° C.

### Experiment 3

Experiment 1 was repeated whereby isosorbide was systematically replaced by neopentyl glycol (NPG). The effect on T_{g} and M̅ₙ is shown in FIG. 1, the lower the isosorbide content the lower the T_{g} value is.

### Experiment 4

Experiment 3 was repeated with 80 and 60 mol % isosorbide, resulting in polyesters 3a and 3b and Experiment 3 was repeated whereby instead of NPG either 1,3-propanediol (PD) or 2,3-butanediol (BD) was used, resulting in polyesters 4a, 4b, 4c and 4d. The results are shown in Table 1. The isosorbide content was kept between 100 and 60 mol %, relative to the total amount of diols present. Again the results show that a higher isosorbide content results in a higher T_{g} value.

TABLE 1
Linear terpolyesters based on succinic acid (SA), isosorbide
(IS) and 2,3-butanediol (BD), 1,3-propanediol (PD) or NPG.

| poly- | feed | T_{g} | Mₙ ¹ | M_{w}/ | AV ² | OHV ³ |
|---|---|---|---|---|---|---|
| ester | composition | [° C.] | [g/mol] | Mₙ | [mgKOH/g] | [mgKOH/g] |
| 4a | SA/IS/BD | 50.6 | 2700 | 1.9 | 0.2 | 48.6 |
| | [1:0.92:0.23] | | | | | |
| 4b | SA/IS/BD | 46.8 | 4600 | 1.6 | 2.0 | 32.0 |
| | [1:0.69:0.46] | | | | | |
| 4c | SA/IS/PD | 45.2 | 2700 | 2.0 | 4.7 | 43.9 |
| | [1:0.85:0.20] | | | | | |
| 4d | SA/IS/PD | 20.6 | 3500 | 1.5 | 1.5 | 37.7 |
| | [1:0.69:0.46] | | | | | |
| 3a | SA/IS/NPG | 47.1 | 3500 | 1.5 | 13.6 | 33.5 |
| | [1:0.80:0.20] | | | | | |
| 3b | SA/IS/NPG | 30.5 | 4300 | 1.5 | 7.5 | 34.0 |
| | [1:0.60:0.40] | | | | | |

¹ Determined by SEC in THF, using polystyrene standards
² Acid value: measure of carboxylic acid functionality
³ Hydroxyl value, measure of hydroxyl functionality

### Example 5

### Synthesis of Polyester Based on Isoidide and a Second Diol

Example 2 was repeated wherein part of the isoidide was replaced by: 2,3-butanediol (BD), 1,3-propanediol (PD) and trimethylolpropane (TMP), respectively, resulting in polyesters 5a, 5b and 5c. The latter monomer led to branched polyesters with an increased OH-functionality. The results show that replacement of the isoidide by an alcohol can be used to control the crystallinity of the polyester.

TABLE 2
Isoidide(II) and succinic acid(SA) based polyesters

| | polyester | Mₙ ¹ | | T_{g} | Tₘ | AV ² | OHV ³ |
|---|---|---|---|---|---|---|---|
| polyester | composition | [g/mol] | M_{w}/Mₙ | [° C.] | [° C.] | [mgKOH/g] | [mgKOH/g] |
| 2 | SA/II | 4300 | 1.9 | 73.4 | 171 | 0 | 35.0 |
| | [1:1.08] | | | | | | |
| 5a | SA/II/BD | 6700 | 2.0 | 51.3 | — | 6.0 | 30.8 |
| | [1:0.89:0.14] | | | | | | |
| 5b | SA/II/PD | 5900 | 2.0 | 43.7 | — | 4.6 | 59.2 |
| | [1:0.82:0.23] | | | | | | |
| 5c | SA/II/TMP | 5200 | 3.4 | 39.7 | — | 5.0 | 104.0 |
| | [1:1.03:0.10] | | | | | | |

¹ Determined by SEC in HFIP, using PMMA standards
² Acid value: measure of carboxylic acid functionality
³ Hydroxyl value, measure of hydroxyl functionality

### Example 6

Polyesters obtained from Examples and Experiments 1 to 5 were cured by using a trimer of hexamethylene diisocyanate (NCO equivalent weight=183 g/mol (trade name: Desmodur N3600, Bayer) according to the following procedure: a solution of 0.3-0.5 g of the polyester in 0.7 mL of NMP was prepared, as well as a separate solution of Desmodur N3600 (1.05 molar equivalent, calculated from titration data) in 0.3 mL of NMP. The two solutions were mixed and applied directly to an aluminum substrate as a wet film with a thickness of 250 μm. After drying at room temperature, the film was cured under N₂. Coating properties are given in Table 3.

The thermal stability of the cured polyesters was investigated by thermogravimetric analysis (TGA). No significant weight loss was observed up to 250° C.

TABLE 3
Coating properties

| | | | | acetone | impact test | König | av. film |
|---|---|---|---|---|---|---|---|
| Poly- | curing | T_{cure} | t_{cure} | resis- | [1 kg, 100 | hard- | thickness |
| ester | agent ^{[1]} | [° C.] | [min] | tance ^{[2]} | cm] ^{[2]} | ness [s] | [μm] |
| 1 | II | 180 | 20 | + | + | 204 | 43 |
| 4a | I | 200 | 30 | − | − | 222 | 40 |
| 4a | II | 180 | 20 | − | +/− | n.d. | 38 |
| 4a | III | 200 | 30 | − | − | 216 | 69 |
| 4c | II | 180 | 20 | − | − | 211 | 27 |
| 4d | II | 180 | 20 | − | + | 61 | 34 |
| 5a | III | 180 | 20 | +/− | +/− | 217 | 40 |
| 5b | II | 180 | 20 | + | + | 215 | 36 |
| 5c | II | 180 | 20 | + | + | 205 | 42 |

^{[1]} I: Vestagon B1530, an ε-caprolactam blocked trimer of isophorone diisocyanate (NCO equivalent weight = 275 g/mol), II: Desmodur N3600, a trimer of hexamethylene diisocyanate (NCO equivalent weight = 183 g/mol), III: Desmodur BL3272, an ε-caprolactam blocked trimer of hexamethylene diisocyanate (NCO equivalent weight = 410 g/mol).
^{[2]} + = good, +/− = moderate, − = poor

1. Process for the production of a polyester by the polycondensation of a mixture comprising isoidide, and a dicarboxylic acid or dicarboxylic acid anhydride, wherein the reaction is performed in the melt of the monomers and wherein these monomers are not activated.

2. Process according to claim 1 characterized in that the polycondensation is performed at a temperature between 150 and 250° C.

3. Process according to claim 1 or 2 characterized in that the polycondensation is performed in a setup which is continuously flushed with an inert gas.

4. Process according to any one of claims 1 to 3 characterized in that the polycondensation is performed in the presence of an esterification catalyst.

5. Process according to claim 4 characterized in that the esterification catalyst is chosen from the group consisting of tetrabutyltitanate, tin(II)octoate, butyltinchloridedihydroxyide, manganese acetate, zink acetate and paratoluene sulphonic acid.

6. Process according to any one of claims 1 to 5 characterized in that the isoidide has a purity between 98 and 100%.

7. Process according to any one of claims 1 to 6 characterized in that as dicarboxylic acid is used an aliphatic di- or polyvalent carboxylic acid having 4 to 20 carbon atoms.

8. Process according to claim 7 characterized in that as dicarboxylic acid is used succinic acid, glutaric acid, adipic acid or sebacic acid, respectively having 4, 5, 6 and 10 carbon atoms.

9. Process according to any one of claims 1 to 8 characterized in that 1-50% of the isoidide is replaced by an alcohol having two or more hydroxyl groups.

10. Process according to claim 9 characterized in that 1,3-propanediol, 1,4-butanediol, 2,3-butanediol and/or trimethylolpropane is used as the alcohol having two or more hydroxyl groups.

11. Polyester obtainable by the process according to any one of claim 1-10.

12. Polyester according to formula (IV) 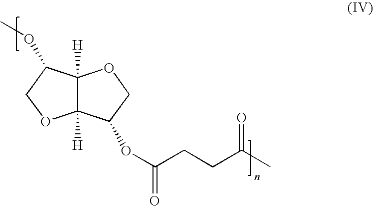
wherein n ranges from 3 to 300

13. Use of a polyester obtainable by the polycondensation of a mixture comprising a dianhydrohexitol, and a dicarboxylic acid or dicarboxylic acid anhydride, wherein the reaction is performed in the melt of the monomers and wherein these monomers are not activated, in a powder paint composition, a toner composition and/or in an engineering plastic.

14. Binder composition comprising a polyester obtainable by the polycondensation of a mixture comprising a dianhydrohexitol, and a dicarboxylic acid or dicarboxylic acid anhydride, wherein the reaction is performed in the melt of the monomers and wherein these monomers are not activated, and a crosslinker.

15. Binder composition according to claim 14 characterized in that as crosslinker the trimer of hexamethylene diisocyanate is applied.

16. Coating composition comprising a binder composition according to claim 14 or 15, and at least one additive.

17. Substrate that is partially or fully coated with a coating composition according to claim 16.

18. Cured coating composition according to claim 16.
